(19) United States
(12) Patent Application Publication
(10) Pub. No.: US 2019/0293649 A1
(43) Pub. Date: Sep. 26, 2019

(54) Proteome Editing System

(71) Applicant: The Government of the United States of America, as represented by the Secretary of the Navy, Arlington, VA (US)

(72) Inventors: Patricia M. Legler, Derwood, MD (US); Elaine Morazzani, Leesburg, VA (US); Pamela Glass, Frederick, MD (US); Jaimee R. Compton, Washington, DC (US)

(21) Appl. No.: 16/413,282

(22) Filed: May 15, 2019

Related U.S. Application Data
(62) Division of application No. 15/820,969, filed on Nov. 22, 2017.
(60) Provisional application No. 62/426,352, filed on Nov. 25, 2016.

(51) Int. Cl. G01N 33/569; C12N 15/11; C12Q 1/37; C12N 7/00; C12N 15/90; C12N 9/96; C12N 9/22

(52) U.S. Cl. CPC G01N 33/56983; C12N 15/11; C12Q 1/37; C12N 7/00; C12N 15/902; C12N 9/96; C12N 2770/36131; G01N 2333/181; Y02A 50/53; Y02A 50/54; C12N 2310/20; C12N 2770/36111; C12N 9/22

(57) ABSTRACT

A protease of the Venezuelan equine encephalitis virus (VEEV) was found to act on a host substrate in addition to the viral substrate. It is contemplated that these findings could be employed to facilitate post-translational silencing at the level of protein (removal of existing proteins) as a protein analog to CRISPR/Cas9 and RNAi/RISC, and further to enable detection of viral infection.

### CROSS-REFERENCE TO RELATED APPLICATIONS

This application claims the benefit of U.S. Provisional Application 62/426,352 filed on Nov. 25, 2016, and is a division of U.S. patent application Ser. No. 15/820,969 file on Nov. 22, 2017, the entirety of each of which is incorporated herein by reference.

### BACKGROUND

Venezuelan equine encephalitis virus (VEEV) is a New World alphavirus. VEEV viral particles are highly resistant to desiccation and can be stably lyophilized and aerosolized (1) which has implications for its use as a potential bioweapon. Inhaled virus can disseminate into the brain via the olfactory neurons (2-4), and symptoms can occur within 28-33 hours in humans (5-8). Acute alphaviral infections are typically resolved by the innate and adaptive immune responses. Only ˜1% of human VEEV infections result in lethal encephalitis; however, neurological symptoms occur in approximately 14% (5;8;9). The other New World alphaviruses, eastern (EEEV) and western (WEEV) equine encephalitis viruses, share high sequence identity (68%) with VEEV, but are significantly more lethal in humans, with mortality rates of 36% and 10%, respectively (2;4;8;10;14 The Old World alphaviruses such as Chikungunya (CHIKV), Sindbis (SINV), and Semliki Forest (SFV) viruses are more commonly associated with fever, arthralgia, skin rashes, and malaise (12). What accounts for the differences in virulence and pathogenicity is not well delineated.

Alphaviruses are known to utilize their nonstructural and structural proteins to suppress the innate immune responses in order to replicate, and the mechanisms of suppression differ among alphaviruses (13;14). Some similarities in virulence may have arisen from genetic recombination events (e.g. WEEV which has EEEV-like encephalogenic properties is thought to have arisen from a SINV-like and EEEV-like ancestor (15)). Virulence differs in host species, as the name suggests the mortality rates of EEV infections are significantly higher for equine than humans and can range from 40-90% (16).

Alphaviruses are (+)ssRNA viruses and belong to the Togaviridae family of Group IV. Group IV contains 33 families and includes the Coronaviridae, Picornaviridae, and Flaviviridae. During alphaviral replication, recognition of double stranded RNA in the cytoplasm by RIG-I or MDA-5 triggers the mitochondrial antiviral signalosome (MAVS) and results in the rapid production of type I interferons (IFN) and proinflammatory cytokines (17;18). IFN plays an important role in limiting acute alphaviral infections (17-19). IFN can protect uninfected cells from infection and create an antiviral state to prevent further alphaviral replication (20). IFN-stimulated genes (ISG) can inhibit the replication of CHIKV, SINV, and VEEV (21-24). Alphaviruses utilize multiple redundant mechanisms to antagonize the IFN response (25). To evade the innate immune responses alphaviruses shut off host cell transcription and translation, typically within hours post-infection (14;23), to prevent the expression of ISG.

The nonstructural proteins (nsPs) play essential roles in replication, but can also play secondary roles in IFN-antagonism. The role of the nsPs in IFN-antagonism can be either enzymatic or non-enzymatic (e.g. binding). The nsP2 of alphaviruses contains an N-terminal domain, a helicase, a papain-like protease, and an S-adenosyl-L-methionine-dependent RNA methyltransferase (SAM MTase) domain (FIG. 1A). The nsP2 of Old World alphaviruses, SINV, SFV, and CHIKV, can inhibit transcription in a manner that is independent of its protease activity, but reliant on its helicase activity (26). These nsP2 proteins induce the rapid degradation of Rpb1, a catalytic subunit of the RNA polymerase II complex, through nsP2-mediated ubiquitination. The ubiquitination of Rpb1 depends on the enzymatic activity of the Old World nsP2 helicase, but also on the integrity of the SAM MTase domain. Mutations within these domains were shown to abolish Rpb1 degradation (26). The transcriptional shut-off mechanisms are known to differ for Old and New World alphaviruses (27). In cells infected with the New World alphavirus, VEEV, transcriptional shutoff is mediated by a 39-residue sequence at the N-terminus of the capsid protein; the capsid is thought to partially obstruct the nuclear pore complex to block host mRNA export (28;29). While these viruses can effectively counter the innate immune responses using these shutoff mechanisms, intrinsic immune factors pose additional challenges since these proteins are present prior to viral infection and sufficient quantities of viral proteins (e.g. capsid) may not be present to override their effects early in infection. Catalytic amounts of the viral enzymes may thus be important for establishing infection.

As described below, the VEEV nsP2 protease was found by the inventors to play a role in interferon antagonism, the mechanism hasimplications with regard totechniques to “silence” expressed proteins. Prior methods to reduce protein concentrations in a cell include CRISPR/Cas9 and RNAi/RISC. Because these methods work at the level of DNA and RNA, respectively, they must be applied prior to protein expression and thus cannot alter the concentrations of proteins that have already been expressed in a cell or have entered into a cell (e.g. protein toxin).

### BRIEF SUMMARY

In one embodiment, a method of detecting infection includes obtaining biological material from an individual suspected of being infected with a Group IV virus; and assaying the biological material to detect the presence or absence of a cleavage product of a protease of the Group IV virus, wherein the presence of a particular host protein cleavage product indicates that the individual is likely infected with a specific Group IV virus. The host protein that is cleaved is specific to the viral protease and can be predicted from sequence homology between the viral protease cleavage site motif sequence and the human host protein.

In a further embodiment, a method of cleaving a desired host protein target includes causing a cell to express a recombinant viral RNA that encodes a cleavage site recognized by a protease (natural or engineered); and infecting the cell with the recombinant Group IV virus, thereby causing the viral protease to cleave the recombinant viral polyprotein and the corresponding target host protein at the cleavage site.

### BRIEF DESCRIPTION OF THE DRAWINGS

FIGS. 1A through 1C illustrate the organization of the alphaviral nonstructural polyprotein. As seen in FIG. 1A, the nonstructural protein 2 (nsP2) contains an N-terminal region, a helicase, a papain-like cysteine protease (white), and SAM methyltransferase (SAM MTase, horizontal stripe). The nsP2 cysteine protease cleaves the polyprotein to produce nsP1, nsP2, nsP3, and nsP4. FIG. 1B shows the crystal structure (PDB 5EZS) (30 of the nsP2 cysteine protease inhibited with E64d. The protease and SAM MTase domains pack together. The peptide-like E64d inhibitor binds beneath the β-hairpin at the interface of these two domains. The structure of the pre-cleavage nsP23 complex (PDB 4GUA) shows the packing of the nsP3 domain (90). FIG. 1C illustrates a sequence alignment of the TRIM14 protein with the three alphaviral nsP cleavage sites used in the substrates. The New World alphaviruses and human TRIM14 share the QEAGA↓G (SEQ ID No: 1) sequence.

FIGS. 2A through 2C show results of in vitro assays demonstrating the cleavage of TRIM14 by the VEEV nsP2 cysteine protease. FIG. 2A shows the results from measurement of the VEEV nsP2 cysteine protease steady state kinetic parameters for the CFP-TRIM14-YFP substrate measured at R.T. in 50 mM HEPES pH 7.0 for 30 min. The Kₘ and Vₘₐₓ were comparable to those measured using similar substrates containing the VEEV nsP12 or nsP34 cleavage sites. FIG. 2B shows results after the VEEV, EEEV, WEEV, or CHIKV nsP2 cysteine proteases (5 μM) were incubated with 50 μM CFP-TRIM14-YFP substrate for 24 h at R.T. in 50 mM HEPES pH 7.0, 150 mM NaCl. Only the VEEV nsP2 cysteine protease was able to digest the TRIM14 substrate completely. FIG. 2C shows the effects of site-directed mutagenesis on cleavage of CFP-YFP substrates containing the SFV nsP12 cleavage site, the VEEV nsP12, nsP23, nsP34 cleavage sites and the TRIM14 sequence. Cleavage reactions were run in 1×PBS pH 7.4 and 5 mM DTT and were incubated for 19 h at R.T. using 30 μM substrate and 2.2 μM enzyme.

FIGS. 3A and 3B illustrates the mass spectra of the CFP-TRIM14-YFP proteolytic products. Proteolytic products of the CFP-TRIM14-YFP 25-residue substrate after cleavage by the VEEV nsP2 cysteine protease were separated by SDS-PAGE, excised, trypsinized, and identified by tandem mass spectrometry to verify the specificity of the protease. Annotated MS/MS spectra of the HYWEVDVQEAGA and GWWVGAMVS are shown. For simplicity only singly charged fragments were annotated. All predicted singly charged fragment ions were found.

FIGS. 4A through 4E show evidence of VEEV nsP2 protease cleavage of TRIM14 in infected cell lysates. Immunoblots of (FIG. 4A) VEEV, (FIG. 4B) WEEV, (FIG. 4C) EEEV-infected cell lysates using an anti-TRIM14 Sigma Prestige polyclonal antibody (HPA053217) that recognizes an epitope common to all 3 isoforms of TRIM14. Cell lysates were removed at various time points (6-96 h). While fluctuations in band intensities were observed during the course of infection, only the VEEV-infected cell lysates produced a new band with a MW consistent with nsP2 cleavage. The cleavage product (CP) band was not detectable in uninfected controls. Multiple bands were observed likely due to the poly-ubiquitination of TRIM14 and the multiple isoforms (α and β) of the protein. FIG. 4D shows the calculated molecular weights (MW) of each isoform and cleavage product. FIG. 4E is a replicate showing the 6 and 24 h time points.

FIG. 5 shows the inhibition of VEEV nsP2 protease cleavage of TRIM14 by CA074. A549 cells were treated with varying concentrations of a nsP2 cysteine protease inhibitor, CA074 methylester (42), and then infected with VEEV. Cell lysates were examined by immunoblot analysis using the anti-TRIM14 antibody HPA053217. The TRIM14 CP was present in the infected cells that had not been treated with the protease inhibitor (labeled NC for “no compound”), and was absent in cell lysates treated with the nsP2 cysteine protease inhibitor. Infection was confirmed by immunoblot analysis using anti-VEEV sera in the lower blot.

FIG. 6 is a partial sequence alignment of the C-terminal domain of TRIM14 homologues from other species. The region shown contains the predicted PRY/SPRY domain of the TRIM14 protein. In gray are the PRY/SPRY domain motifs (“LDP”, “WEVD”, “LDYE”) (91). The QEAGA↓G (SEQ ID No: 1) motif is shown in bold. In human TRIM14 Lys-365 (highlighted) was shown to be poly-ubiquitinated. This ubiquitination site is important for recruitment of NEMO to the MAVS signalosome.

FIG. 7 illustrates how several Group IV (+)ssRNA viral proteases cleave components of the MAVS signalosome. The MAVS signaling cascade proposed by Zhou, et al is shown (32). The MAVS signalosome triggers the production of IFN and pro-inflammatory cytokines. The VEEV nsp2 cysteine protease cleavage site in TRIM14 is located before the ubiquitination site. Cleavage of the proteins involved in the signalosome would likely disrupt the production of IFN and the innate immune response.

FIG. 8 illustrates three mechanisms of silencing (based on DNA, RNA, and protein) that are guided by a short sequence. In each case a short sequence is used to identify a larger target sequence; these mechanisms are analogous to search and delete programs that utilize a keyword and have been written in three different languages. Each system has an enzyme that recognizes the match between the short sequence and the target, and then cuts the larger target sequence. The short sequence and target sequences belong to either the host or pathogen, and the goal of these mechanisms is to antagonize or silence the effects of the molecule. These mechanisms are used to defend the host from viruses, or to defend a virus from a host's immune system. The CRISPR/Cas9 and RNAi figures have been adapted from ref. (92) and ref. (93).

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

As used herein, “suspected of being infected” is meant to be interpreted very broadly to compass instances where an infection is virtually certain to those where it is not believed that an infection exists.

### Overview

The alphaviral nonstructural protein 2 (nsP2) cysteine proteases (EC 3.4.22.-) are involved in the proteolytic processing of the nonstructural (ns) polyprotein. After examining the substrate specificities of the VEEV nsP2 cysteine protease, a new host substrate of the VEEV nsP2 protease, human TRIM14, was identified. The TRIM14 protein is a component of the mitochondrial antiviral-signaling protein (MAVS) signalosome. The same amino acid sequences, termed short stretches of homologous host-pathogen protein sequences (SSHHPS), are present in both the nonstructural polyprotein and TRIM14

It is contemplated that these findings could be employed to facilitate post-translational silencing at the level of protein (removal of existing proteins) as a protein analog to CRISPR/Cas9 and RNAi/RISC. This system relies on the SSHHPS and a protease (as opposed to a nuclease) that cleaves them. It is further contemplated that the presence or absence of a viral infection could be detected by analysis of the cleavage products of the nsP2 protease and similar proteases, or the consequent downstream effects produced from silencing a signaling cascade using the nsP protease.

### Description

The present inventors hypothesized that the alphaviral protease cleavage sites may share homology to human proteins and that the virus may use these short stretches of host sequences in its cleavage sites as another mechanism of IFN-antagonism. The VEEV nsP2 substrate specificities were previously characterized using kinetic, mutational and structural studies (30. The inventors examined potential host protein targets of the nsP2 protease by searching the human genome for proteins sharing sequence identity with the nsP12, nsP23, and nsP34 cleavage site sequence motifs. One human protein, TRIM14 (also known as Pub (31)), sharing six identical residues to an alphaviral nsP12 cleavage site, is a substrate of the VEEV nsP2 viral protease. Consistent with in vitro assay results—TRIM14 cleavage could be detected in immunoblots of VEEV-infected cell lysates.

TRIM14 is a tripartite motif protein (TRIM) and was recently shown to function as an adaptor protein in the MAVS signalosome (32;33). Stable overexpression of TRIM14 has been shown to inhibit alphaviral replication by 3-4 logs 24 h post-infection using SINV (34). TRIM14 overexpression also increased the transcription of IFNs and interferon stimulated genes (33). The viral proteases' ability to cleave a protein involved in the production of IFN appears to be a common antagonistic mechanism used by this and other Group IV viral proteases. We discuss the similarities of this silencing mechanism with those of CRISPR/Cas9 and RNAi/RISC

At least eight other Group IV (+)ssRNA viral proteases have been shown to cleave components of the MAVS signalosome to antagonize IFN production suggesting that the assimilation of these short cleavage site motif sequences to host protein sequences may represent an embedded mechanism of IFN antagonism. This interference mechanism shows several parallels with those of CRISPR/Cas9 and RNAi/RISC, but with a protease recognizing a protein sequence common to both the host and pathogen.

### EXAMPLES

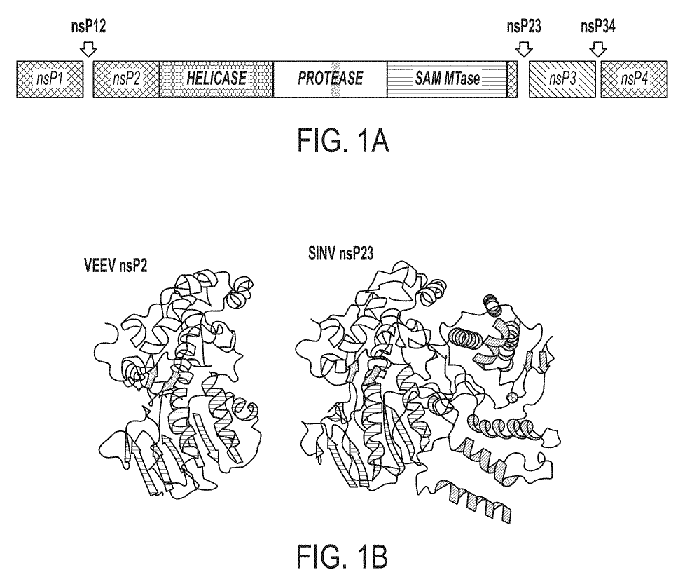

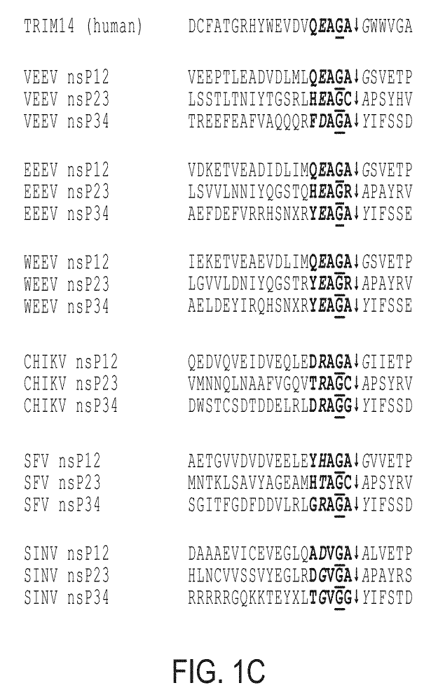

The sequences N- and C-terminal to the scissile bond that were recognized by the VEEV nsP2 cysteine protease were previously identified using a set of peptide substrates. The 25-residue substrates containing P19-P6′ (Schechter and Berger nomenclature (35)) produced the lowest Kₘ values (30). A BLAST search (36) using the nsP2 cleavage sites and the human genome uncovered one protein, TRIM14, which had a high level of sequence identity to the VEEV nsP12 cleavage site. The nsP12 cleavage site QEAGA↓G (SEQ ID No: 1) is highly conserved among the more virulent New World alphaviruses, VEEV/EEEV/WEEV, but not in the Old World alphaviruses such as SINV, SFV, and CHIKV (FIG. 1C). Some overlap in substrate specificities has been observed for the VEEV and CHIKV nsP2 proteases; both are able to cleave the Old World SFV nsP12 cleavage site (30).

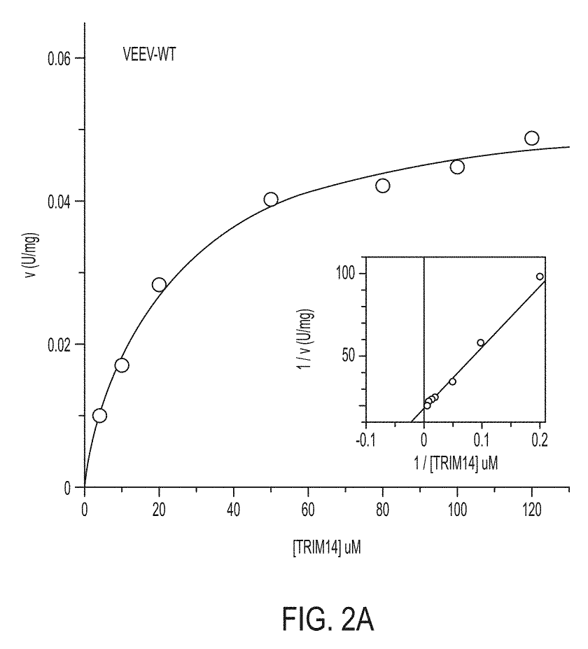

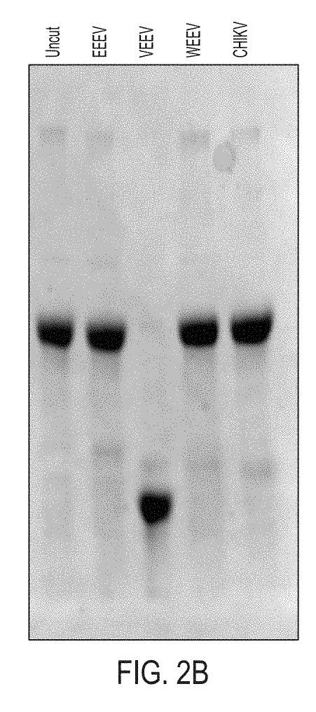

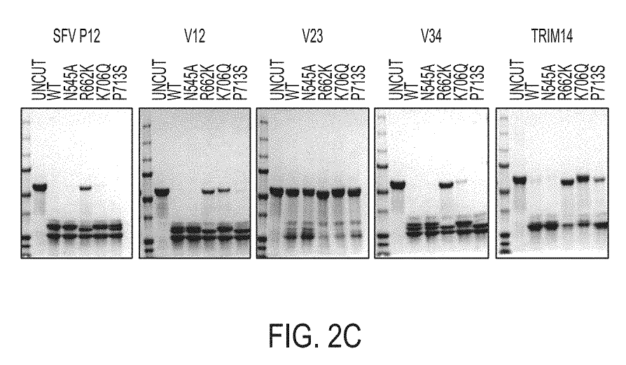

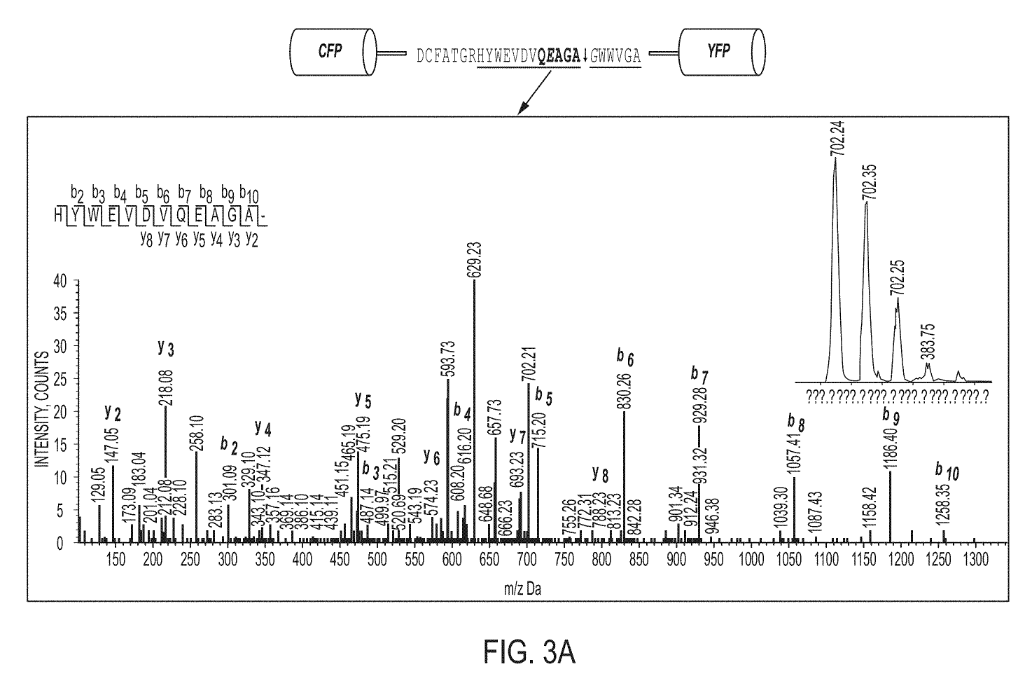

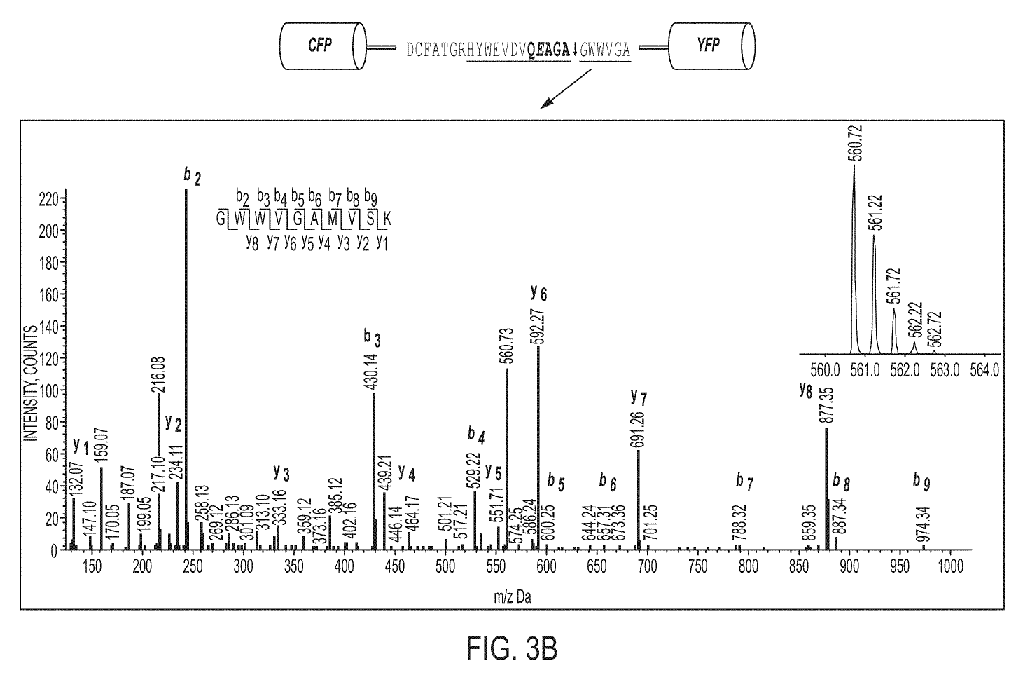

Using a cyan and yellow fluorescent protein (CFP-YFP) substrate containing 25-amino acids of the human TRIM14 protein, the purified VEEV nsP2 protease was found to cleave the TRIM14 substrate (FIG. 2A), but no cleavage occurred with related viruses (FIG. 2B). Cleavage was confirmed by SDS-PAGE, and the effects of site-directed mutagenesis on the cleavage of the TRIM14 substrate were similar to those observed for the substrate containing the VEEV nsP12 cleavage site suggesting similar enzyme and substrate contacts (FIG. 2C). For the VEEV nsP2 protease, the cleavage site in the CFP-YFP substrate was confirmed by tandem mass spectrometry (FIG. 3), and cleavage occurred at the expected site at QEAGA↓G (SEQ ID No: 1). The tryptic peptides of both parts of the substrate were identified.

Steady state kinetic parameters were measured to determine if the Kₘ and Vₘₐₓ measured with the TRIM14 25-residue substrate were similar to those obtained with substrates containing the viral cleavage sites (FIG. 2A). The Kₘ and Vₘₐₓ obtained with the wild type (WT) VEEV nsP2 protease and the TRIM14 substrate were comparable to those obtained with the nsP12 and nsP34 substrates. The length of the substrate was also varied and 25-, 22-, and 19-residue substrates were tested (Table 1). As the length of the region N-terminal to the scissile bond decreased, an increase in the Kₘ and Vₘₐₓ was observed consistent with weaker binding and faster product release.

To determine if the cleavage was specific to the VEEV nsP2pro, the proteases of VEEV, EEEV, WEEV and CHIKV were expressed and purified. With the 25-residue TRIM14 substrate, complete cleavage of the substrate (50 μM) by the VEEV protease (5 μM) was visible after 24 h at 23±3° C. by SDS-PAGE (FIG. 2B); however, with the shorter TRIM14 substrates the purified CHIKV, EEEV, and WEEV nsP2 proteases only produced low levels of cleavage product even after extensive incubation (64 h, 23±3° C.) (VEEV>WEEV>EEEV>CHIKV) (data not shown). The corresponding viral cleavage sites were also digested for relative comparison since these proteases differ in activity. All four proteases had detectable activity. Only the VEEV nsP2 cysteine protease consistently cut all of the TRIM14 substrates.

TABLE 1
Steady state kinetic parameters for the VEEV nsP2 cysteine protease
measured in 50 mM HEPES pH 7.0 at room temperature (R.T.).

| | | | Vₘₐₓ | Kₘ |
|---|---|---|---|---|
| Substrate | | Length | (U/mg) | (μM) |
| CFP-V12-YFP | VEEPTLEADVDLMLQEAGA↓GSVETP | 25 | 0.059 ± 0.003 | 12 ± 3 |
| | (SEQ ID No: 2) | | | |
| CFP-V34-YFP | TREEFEAFVAQQQRFDAGA↓YIFSSD | 25 | 0.089 ± 0.005 | 21 ± 4 |
| | (SEQ ID No: 3) | | | |
| CFP-TRIM14-YFP | DCFATGRHYWEVDVQEAGA↓GWWVGA | 25 | 0.056 ± 0.002 | 21 ± 2 |
| | (SEQ ID No: 4) | | | |
| | ATGRHYWEVDVQEAGA↓GWWVGA | 22 | 0.0080 ± 0.0003 | 26 ± 4 |
| | (SEQ ID No: 5) | | | |
| | RHYWEVDVQEAGA↓GWWVGA | 19 | 0.012 ± 0.002 | 50 ± 20 |
| | (SEQ ID No: 6) | | | |

A computer model was created of the binding interactions of TRIM14 with the VEEV nsP2 cysteine protease in order to gain insight into the structural basis of substrate specificity. Like the New World alphaviral substrates, TRIM14 contains a Glu at position P4 which may explain why no cleavage of TRIM14 was observed with the Old World CHIKV nsP2 protease. In the nsP12 cleavage site, the P1′-P6′ residues are identical in sequence for VEEV/EEEV/WEEV, as are the P1-P5 residues. This suggests that residues beyond P5 are important for recognition of the TRIM14 substrate. To understand why the 25-amino acid substrate led to the lowest Kₘ and highest k_{cat}, we examined our previously determined crystal structure of the free VEEV nsP2 protease, PDB 5EZQ (30. The crystal structure contains the C-terminal P2-P19 residues (Leu-776-Ala-792) of the VEEV nsP23 cleavage site; the P10-P19 residues are helical and are packed against the protease domain in the crystal. The P8-P9 residues are directed into the cleft formed by the protease and SAM MTase domains (data not shown). Chou-Fasman secondary structure predictions suggest that the nsP12 and nsP34 substrates may contain helical regions within the P1-P19 residues.

Regions beyond P5 were examined to understand why the EEEV and WEEV enzymes cut TRIM14 poorly. Based on the Kₘ values (Table 1) the P13-P19 residues of the substrate appear to make additional contacts to the enzyme. In PDB 5EZQ the P17 residue (Ser-778) within the helix of the symmetry related molecule is within hydrogen bonding distance to the backbone NH and C═O of the papain-like protease domain residue Met-555. Met-555 is conserved in the VEEV/EEEV/WEEV nsP2 cysteine proteases. The P19-P16 residues of the substrates differ in charge and flexibility in the New World polyproteins and may be recognized differently by these closely related proteases: “VEEP” in VEEV nsP12; “VDKE” in EEEV nsP12; and “IEKE” in WEEV nsP12. The homologous residues in TRIM14 are “DCFA.”

Cleavage of the TRIM14 substrate by mutants of the protease was examined to confirm the models of the VEEV nsP2 cysteine protease (FIG. 2C). The K706Q mutation affected the cleavage of V12 and TRIM consistent with the disruption of substrate binding interactions in the predicted S4 subsite. The P4 residues (Glu) are the same in both of these substrates. The purity of the VEEV nsP2 protease was also examined using the C477A variant. Strauss et al. had previously shown that the nonstructural polyprotein was not cut by any host enzymes in eukaryotic cells (37); similarly, non-specific cleavage of these protein substrates was not observed with the CFP-YFP substrates expressed and purified from E. coli.

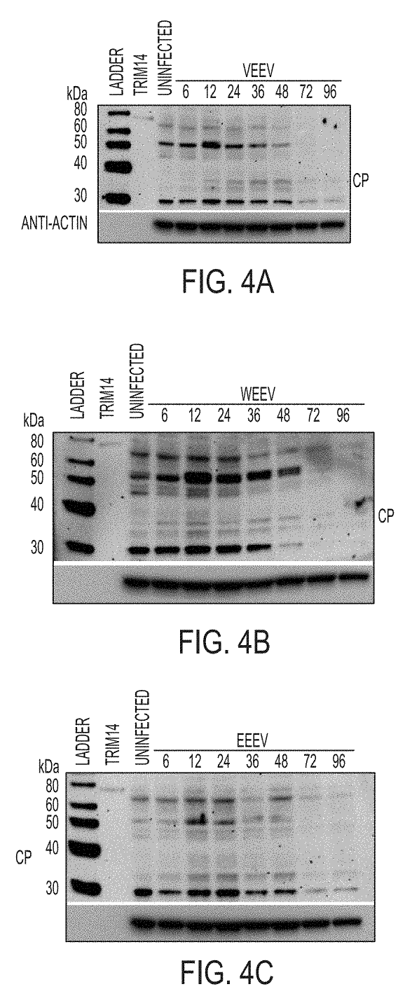

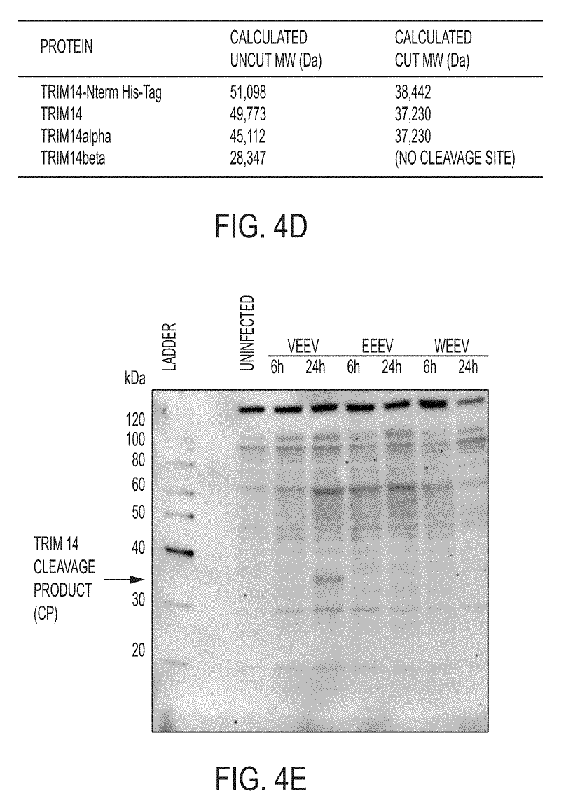

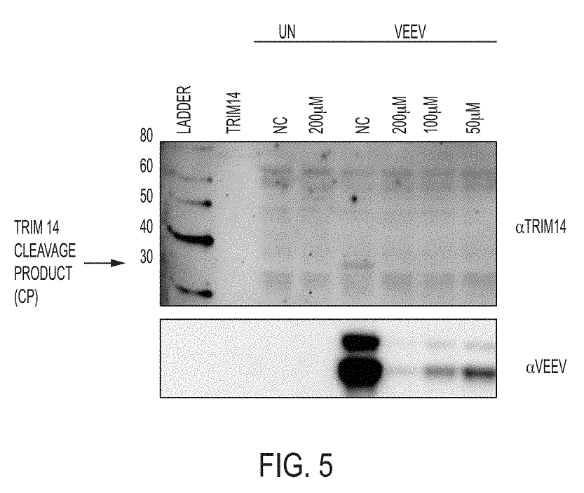

Sequence alignment analysis showed that full length TRIM14 (442 amino acids, 49.8 kDa) and the TRIM14-α isoform (406 amino acids, 45.1 kDa) contain the cleavage site while the TRIM14-β isoform (28.3 kDa) does not. TRIM14 was shown to be poly-ubiquitinated at K48 and K63 (32), and multiple bands were detected in immunoblots (FIGS. 4A, 4B, 4C). The anti-TRIM14 antibody used in this work is a Sigma Prestige™ antibody (HPA053217) that has been previously validated and shown to be specific for its antigen in cell lysates and peptide libraries; characterization of this antibody can be found in the Human Protein Atlas (38).

The calculated molecular weights of unmodified TRIM14 cleavage products are 37.2 kDa and 12.6 kDa (or 7.9 for the TRIM14a isoform). The recombinant TRIM14 used as a control in the immunoblots is a GST-fusion protein (˜76 kDa). It is important to note that the stability of the cleavage products in cells is unknown, and quantitative conclusions are limited using cell lysates (e.g. calculation of the percentage of TRIM14 cleaved in virus infected cells). TRIM14 is polyubiquitinated at K48 for degradation (39) and at K63 to facilitate its role in signaling (32). Overexpression of TRIM14 has been shown to suppress alphaviral replication (33) and hepatitis C replication (40.

TRIM14 cleavage in VEEV-infected cells was monitored over time, and cell lysates were collected at 6, 12, 24, 36, 48, 72, and 96 hours. The band intensities varied over time; however, only the VEEV- and WEEV-infected cell lysates contained a new ˜37 kDa cleavage product that was not found in the uninfected controls (FIGS. 4A and 4B). The 50 kDa band intensified during infection and may be due to enhanced expression of the TRIM14 during viral infection or release from a larger complex. The MW of the cleavage product was consistent with the calculated MW and with the in vitro results using purified recombinant nsP2 proteases and the 25-, 22-, and 19-residue CFP-YFP TRIM14 substrates. The result also suggests that TRIM14 can be cleaved prior to ubiquitination since the cleavage product corresponds to the MW of the non-ubiquitinated protein.

TRIM14 expression can be detected in the absence of virus (32) indicating that this protein is an intrinsic immune response effector protein. TRIM14 expression can also be further induced by IFNs and can also be considered as an innate immune response effector (41). Upon viral infection Lys-63-linked polyubiquitination of TRIM14 at Lys-365 occurs and was shown to be important for the assembly of the MAVS signalosome (32). Thus, cleavage of the unmodified TRIM14 may interfere with the assembly of the MAVS signalosome.

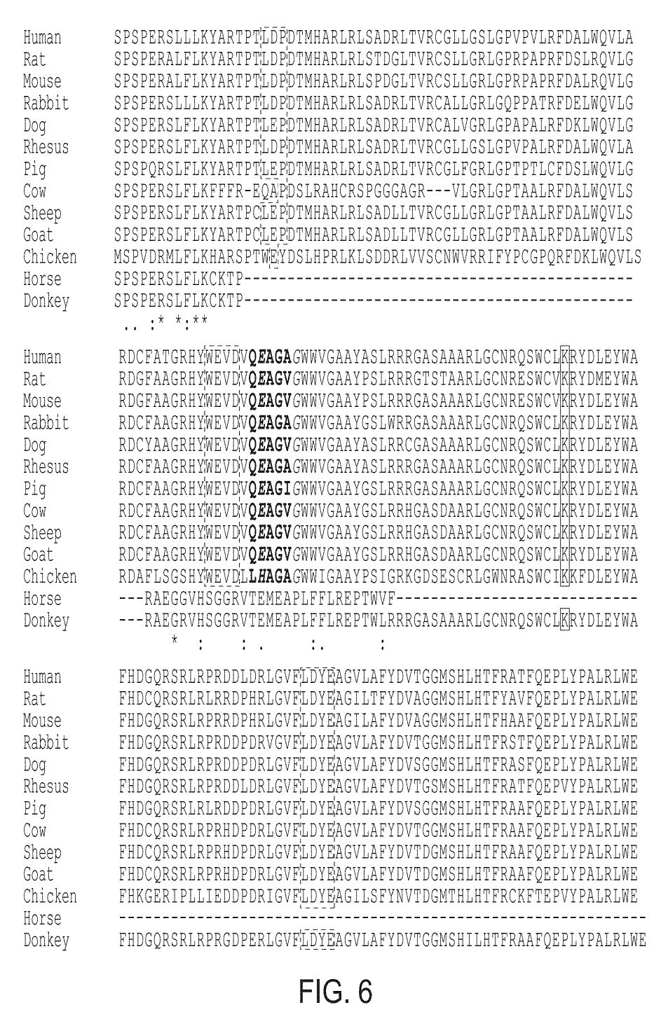

CA074 methyl ester (CA074me) was previously shown to inhibit the alphaviral VEEV nsP2 cysteine protease (42). CA074me is a Cathepsin B inhibitor; however, no other host enzymes have been shown to cleave the nonstructural polyprotein (37). CA074 is a peptide-like irreversible covalent inhibitor that specifically reacts with the nucleophilic Cys of the proteases. CA074me is the membrane permeable form of the inhibitor (prodrug). CA074me was added to cells that were infected with VEEV, and cell lysates were collected and subjected to immunoblotting. The TRIM14 cleavage product was no longer present in the CA074me-treated cells consistent with inhibition of the VEEV nsP2 cysteine protease (FIG. 6).

For acute viral infections, species-specific anti-viral enzymes and proteins that interfere with and counteract viral replication (sometimes referred to as viral restriction factors) exist. One domain within TRIM14 appears to be important to its anti-viral functions and may account for species-specific anti-alphaviral responses (40). Human VEEV infections rarely result in lethal encephalitis (˜1% of infected humans), whereas mortality rates in equine are significantly higher (e.g., EEEV's mortality rate can be as high as 90%) suggesting an inherent difference between the innate immune responses of equid vs. humans. Comparison of TRIM14 homologues from various species shows strong conservation of the full length TRIM14 sequence in humans, monkeys, rodents, pigs, cows, and chickens (FIG. 6). The C-terminal region of equine TRIM14 is notably truncated, indicating that equines may harbor a truncated TRIM14 homologue. The C-terminal region was predicted to form a PRY/SPRY domain. The VEEV nsP2 cysteine protease cleavage site is within this predicted domain. The SPRY domain is a β-stranded protein interaction module commonly found in human proteins that regulate innate and adaptive immunity (43); the PRY motif consists of 3 additional β-strands N-terminal to the SPRY domain. PRY/SPRY domains contain hypervariable loop regions and a conserved core similar to a variable domain of an antibody (44). The binding specificity of the SPRY domain determines the function of the TRIM protein, and mutations within this domain have been associated with disease susceptibility (44). This domain appears to be important for mounting an effective immune response against alphaviruses, as well as HCV (40). The transient proteolytic cleavage of the PRY/SPRY domain during infection, or the absence of this domain as in the case of equine TRIM14, may impair a species' ability to mount an effective antiviral immune response to alphaviruses.

PRY/SPRY domains can be identified by 3 highly conserved sequence motifs (“LDP”, “WEVD/E”, “LDYE/D”). These three motifs are present in the human TRIM14 homologue, but are absent from the equine TRIM14 homologue (FIG. 6). Interestingly, the donkey homologue contains the “LDYE” motif, but lacks the other two motifs. The presence or absence of the PRY/SPRY domain of TRIM14 was not sufficient to predict the virulence or pathogenicity of VEEV in other species; e.g., VEEV infections can be lethal in mice and the murine TRIM14 contains the PRY/SPRY domain. The role of TRIM14 and the downstream effectors (e.g, IFN-stimulated genes, ISG) of this pathway have not been examined across species and may differ. Species-specific differences in the Jak/STAT pathway, a pathway triggered by type I IFN, also cannot be excluded.

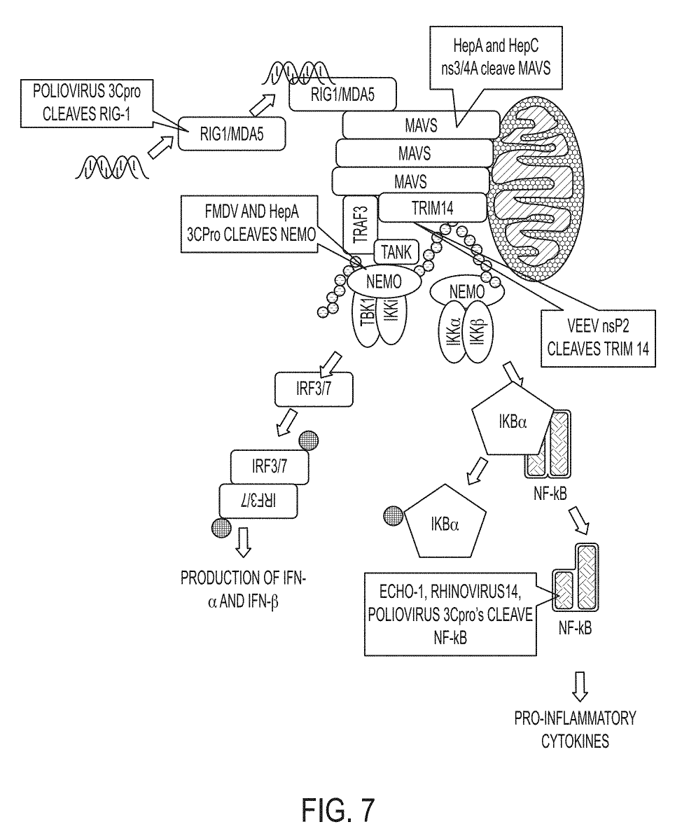

The PRY/SPRY domain is thought to mediate the association of TRIM14 to the C-terminal domain (residues 360-540) of MAVS (32) (FIG. 7). TRIM14 undergoes ubiquitination at a site within the PRY/SPRY domain at Lys-365 and recruits NF-KB essential modulator (NEMO) to activate the IFN regulatory factors 3 and 7 (IRF-3/7) and NF-κB pathways (32). The ubiquitination of Lys-365 was shown to be critical for the association of NEMO to the MAVS signalosome by Zhou et al. (FIG. 7). Phosphorylation of IRF-3 leads to the production of type I IFNs. The VEEV nsP2 cysteine protease cleavage site is 31 residues before Lys-365, and cleavage likely short circuits this cascade to prevent the downstream effects.

Discussion

The proteolytic cleavage of components of the MAVS signalosome by viral proteases appears to be a common mechanism for innate immune response evasion by Group IV (+)ssRNA viruses (Table 2), but has also been observed with other viruses (e.g. influenza (55)). Viral proteases can directly cleave host proteins that lead to IFN and ISG production. Cleavage of several of the targets facilitates the shutoff of host transcription and translation. For example the 3Cpro of viruses belonging to Picornaviridae have been shown to cleave RNA polymerase II transcription factors, TATA-binding protein (56;57), CREB (cAMP responsive element binding protein), Oct-1, p53, SL-1 TBP-associated factors (58), poly(A)-binding protein (59;60), eIF5B (61), eIF4AI (62), eIF4GI (63), TRIF (64), RIG-I (65), MDA-5 (66), MAVS (67) NF-κB (68), and NEMO (69;70). The Hepatitis C (HCV) viral ns3/4A protease (Flaviviridae) was shown to cleave MAVS (71-74). Here we have shown that the VEEV nsP2 protease (Togaviridae) can cleave TRIM14. TRIF (TIR-domain-containing adapter inducing interferon-0) was another common target of viral proteases. The Dengue virus ns2B/ns3 protease was shown to cleave STING (stimulator of the interferon gene, also known as a MITA, mediator of IRF3 activation)(75), a protein that can interact with RIG-I and MAVS, but not with MDA-5. Cleavage of STING led to the inhibition of type I IFN production (75-77). Zika is another notable member of Group IV; however, host proteins that are cleaved by its viral protease have not yet been reported.

The characteristic cleavage products of viral proteases may also produce valuable biomarkers of viral infection and could be useful in the evaluation of the therapeutic efficacy of antiviral protease inhibitors in vivo. For example, MAVS cleavage products were observed in humans with chronic HCV infections, but not in controls, and the cleavage of MAVS by the HCV ns3-4A protease was associated with higher viral loads (73). Since biomarkers for alphaviral infections are relatively uncharacterized, the cleavage of TRIM14 or the downstream effects of cleavage, or both, may be useful indicators of VEEV infection.

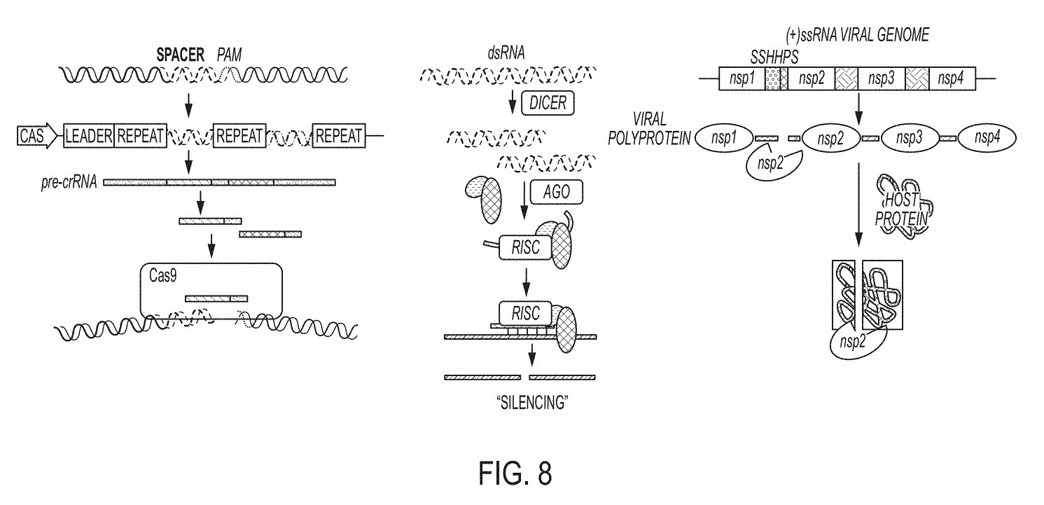

The cleavage of human host proteins by viral proteases has been previously recognized by others (56; 65; 66; 69; 78-83) and may reflect a general antagonistic strategy akin to CRISPR/Cas9 and RNAi/RISC (FIG. 8). The cleavage site sequences recognized by viral proteases do not appear to be randomly selected (Table 2). Several groups have shown that viral proteases can cleave host proteins at sites with relatively little sequence identity to the protease cleavage site sequence in the viral polyprotein. The case presented here shows the longest continuous stretch of identical residues (Table 2). The use of this mechanism by Group IV (+)ssRNA viruses may be due to the translation of the viral genome which is essentially a messenger RNA. The production of viral enzymes, including the RNA-dependent RNA polymerase, precedes the production of dsRNA intermediates. Thus, these viral proteases may have an opportunity to short circuit the MAVS signalosome before the intracellular antiviral responses are triggered by dsRNA intermediates.

A protein version of CRISPR/Cas9 and RNAi/RISC has not been previously described, but could rely on short stretches of homologous host-pathogen protein sequences (SSHHPS) and a protease that cleaves them. By assimilating the relatively short viral protease cleavage sites (˜25 residues) to those of an antiviral intracellular host protein, the virus may effectively gain a function without incorporating a significant amount of new genomic material. The strategy used by these viruses embeds another mechanism of IFN-antagonism reliant on the enzymatic activity of the viral protease (an enzyme that is typically essential for viral replication). Since viruses co-evolve with their hosts, the use of these host protein sequences in the nonstructural protein cleavage sites may have been evolutionarily advantageous since viral replication hinges on the protease. Better suppression of the host's innate immune responses would favor viral replication and could increase the fitness of the virus.

What is common among these three mechanisms of silencing is that they each rely on a short sequence to identify a larger target sequence to destroy; they are analogous to search and delete algorithms that utilize a “keyword” to identify a file to delete (FIG. 8). Each of these programs carries an enzyme able to identify a match between the short sequence and the larger sequence and then cleave the identified target. All of the mechanisms are used to silence or antagonize a response, and the relationship between the short sequence and the target sequence is typically between a host and pathogen, more specifically a virus. Last, these mechanisms are used as defense mechanisms and protect the host from viruses, or a virus from a host. In each case the “keywords,” or an enzyme able to generate a short sequence (e.g. Dicer), were found with the enzyme responsible for cleavage of the target sequence.

Materials and Methods

Materials.

RIPA buffer, Halt™ Protease Inhibitor Cocktail and all general chemicals were purchased from Fisher Scientific (Waltham, Mass.). Plasmid constructs were synthesized by Genscript USA, Inc. (Piscataway, N.J.). BugBuster™ and IPTG (420291) were purchased from EMD Millipore (Bilerica, Mass.). Column resins and PD-10 gel filtration columns were purchased from G. E. Healthcare (Marlborough, Mass.). EDTA-free Protease inhibitor tablets were from Roche, Inc. Black half-area Corning 3993 non-binding surface 96-well plates were from Corning Inc. (Corning, N.Y.). Pierce Precise Tris-HEPES acrylamide gels (8-16% gradient) and BupH Tris-HEPES SDS-PAGE running buffer were from Thermo Scientific (Rockford, Ill.). The anti-TRIM14 antibody (HPA053217), the anti-actin antibody (A1978) and secondary HRP-conjugated antibodies were from Sigma (St. Louis, Mo.).

Plasmid Constructs of FRET Substrates.

A pET-15b plasmid (Ampicillin®) encoding cyan fluorescent protein (CFP), an nsP2 protease cleavage site motif, AG(A/C)↓G/Y/A), and yellow fluorescent protein (YFP) in between the NdeI and XhoI cut sites were synthesized. An N-terminal hexa-histidine tag preceded a thrombin cleavage site. Six CFP-YFP constructs were used: V12 which contains 25-residues of the VEEV nsP12 cleavage site; V34 which contains 25-residues of the VEEV nsP34 cleavage site; S12 which contains 25-residues of the SFV nsP12 cleavage site; and ones containing 25-, 22-, or 19-residues of human TRIM14.

The nsP2 cysteine protease-SAM MTase of CHIKV in a modified pMCSG9 vector (84) was provided by Dr. Jonah Cheung at the New York Structural Biology Center. The CHIKV protease/SAM MTase were fused to a decahistidine-tagged maltose-binding-protein at the N-terminus that could be cleaved using TEV protease

Expression & Purification of the nsP2 Cysteine Proteases.

To ensure purification of the reduced state of the VEEV nsP2 cysteine protease (85), we used an nsP2-thioredoxin (Trx) fusion protein containing the protease and SAM MTase domains (residues 457-792). The EEEV and WEEV nsP2 cysteine proteases were expressed and purified using a similar protocol with an additional Q-Sepharose column purification step prior to the SP-Sepharose column. BL-21(DE3) pLysS E. coli were transformed with the Trx-VEEV-nsP2 plasmid. Luria Bertani (LB) media (3-6 L) containing 50 μg/mL ampicillin and 25 μg/mL chloramphenicol was inoculated and grown to an OD₆₀₀ of approximately 1.0 and induced with 0.5 mM IPTG overnight at 17° C. Cells were pelleted and lysed with lysis buffer (50 mM Tris pH 7.6, 500 mM NaCl, 35% BugBuster, 5% glycerol, 2 mM β-mercaptoethanol (BME), 25 U of DNase 0.3 mg/mL lysozyme) and sonicated ten times for 15 second intervals in an ice bath. Lysates were clarified by centrifugation at 20,000×g for 30 minutes and loaded onto a nickel column equilibrated with 50 mM Tris pH 7.6, 500 mM NaCl, 2 mM BME, 5% glycerol. The column was washed with the same buffer containing 60 mM imidazole. Protein was eluted using the same buffer containing 300 mM imidazole. Protein was dialyzed with thrombin (overnight at 4° C.) against 50 mM Tris pH 7.6, 250 mM NaCl, 5 mM DTT, 1 mM EDTA, 5% glycerol, and then diluted 1:3 with Buffer A (50 mM Tris pH 7.6, 5% glycerol, 5 mM DTT) and loaded onto an SP-Sepharose column equilibrated with Buffer A. Protein was eluted using a salt gradient (0-1.25 M NaCl) and then concentrated, flash frozen in liquid nitrogen, and stored at −80° C. or stored at −20° C. in buffer containing 50% glycerol. The buffer was exchanged to the corresponding assay buffer (50 mM HEPES pH 7.0) prior to all kinetic experiments using PD-10 columns. The CHIKV nsP2 protease was expressed from a construct produced by Chung et al. (86) and was purified using a similar method; the His-tag and MBP were removed.

Expression & Purification of FRET Protein Substrates.

BL-21(DE3) E. coli were transformed with the plasmids encoding the substrates. LB/Amp (1.5 to 3.0 L) was inoculated and grown to an OD₆₀₀ of approximately 1.0 and induced with 0.5 mM IPTG overnight with shaking at 17° C. Cells were pelleted by centrifugation, lysed with lysis buffer (50 mM Tris pH 7.6, 500 mM NaCl, 35% BugBuster, 2 mM BME, 0.3 mg/mL lysozyme, 1 EDTA-free protease inhibitor tablet), and briefly sonicated for 1 minute in an ice bath. Lysates were clarified by centrifugation (20,500×g for 30 minutes at 4° C.) and loaded onto a nickel column equilibrated with 50 mM Tris pH 7.6, 500 mM NaCl, 2 mM BME. The column was washed with the same buffer after loading and with 10-20 column volumes of buffer containing 60 mM imidazole until the A₂₈₀ returned to baseline. The protein was eluted with the same buffer containing 300 mM imidazole. The protein was dialyzed against 50 mM Tris pH 7.6, 150 mM NaCl overnight at 4° C. with 50 U thrombin. The His-tag was removed by re-running the protein on a nickel column and collecting the flow-through. The protein was then dialyzed against 50 mM Tris pH 7.6, 5 mM EDTA, 250 mM NaCl (overnight at 4° C.), followed by dialysis against 50 mM Tris pH 7.6 (2 hours). Protein was loaded onto a Q-Sepharose column equilibrated with 50 mM Tris pH 7.6 and eluted with a salt gradient (0 to 1 M NaCl). All substrates were produced in high yield (typical yields were 60-80 mg per liter of media) and could be readily concentrated to 9.0-10.5 mg/mL. The substrates were used for continuous and discontinuous assays. Similar substrates have been used to study other proteases (87;88).

Continuous FRET Assay.

For measurement of steady state kinetic parameters the method described by Ruge et al. was followed (88). Cleavage of the YFP/CFP FRET substrates was monitored continuously at room temperature (23±3° C.) using excitation/emission wavelengths of 434/470 nm and 434/527 nm to calculate emission ratios and a SpectraMax M5 plate reader from Molecular Devices. The substrate was buffer-exchanged into 50 mM HEPES pH 7.0. Enzyme concentrations of ≤1 μM and a substrate concentration range of 10-140 μM (8 different concentrations) were used to measure Steady State kinetic parameters. Data were collected in triplicate (50 μL reaction volumes) in half-area black low binding surface 96-well plates from Corning, Inc. After the reads were completed the plates were sealed with film and allowed to digest overnight at room temperature 23±3° C. Final emission ratios were read the next day. The fraction of substrate cleaved, f, was calculated from the emission ratios at each time point using the following equation:

$$f = \frac{[ \frac{( \frac{ex 434}{em 527} )}{( \frac{ex 434}{em 470} )} - {r}_{uncut} ]}{( {r}_{cut} - {r}_{uncut} )}$$

The nmols of substrate cleaved at each time point was calculated by multiplying f by the nmols of substrate at t=0 (Sₒ). The value of r_{uncut} corresponds to the emission ratio measured in the absence of enzyme, and the value of r_{cut} is the emission ratio measured when the substrate was fully cleaved. Initial velocities were calculated at each [S] concentration from the linear range (f≤20%). Plots of time vs. nmols were linearly fit for each [S] concentration, and vₒ was obtained from the slopes of the lines. Rates of spontaneous hydrolysis were measured in the absence of enzyme and were subtracted from the enzyme catalyzed rates. Data were fit to the Michaelis-Menten equation, vₒ=(Vₘₐₓ*[S])/(Kₘ+[S]), using Grafit (Erithricus Software Ltd., Surrey, UK).

Discontinuous Gel-Based Assay.

Reaction mixtures (5 μM nsP2-Trx, 50 μM FRET substrate, 50 mM HEPES pH 7.0, 150 mM NaCl) were incubated overnight (˜18 h) at room temperature (23±3° C.). The reactions were run until >90% of the substrate was cleaved by the enzyme. Reactions were stopped by mixing with Laemelli buffer (1:1) and heating the samples for 3 minutes at >70° C. Cleavage products (10 μL) were separated by SDS-PAGE in 12-well 8-16% gradient gels in BupH running buffer (100 mM Tris, 100 mM HEPES, 3 mM SDS, pH 8±0.5) at 110 V for 50 minutes. The calculated molecular weight of the uncut TRIM14 FRET substrate containing a 25 amino acid cleavage sequence was 56.7 kDa, and 29.2 kDa and 27.5 kDa for the cut CFP and YFP products, respectively. The molecular weight of the enzyme for the thioredoxin-His-tagged enzyme was 52.208 kDa, and 38.29 kDa for the Tag-free enzyme. The bands were well separated in 8-16% gradient gels, and boiling of the samples was required to achieve the sharp banding pattern. Densitometry was done using the BioRad Gel Dock Imager software (BioRad Inc., Hercules, Calif.).

Mass Spectrometry.

Gel bands were washed with 250 mM ammonium bicarbonate in 50% acetonitrile (ACN) until completely destained. Bands were then cut into small cubes and dehydrated by 100% (ACN). Modified porcine trypsin solution (Promega, product no. V511) in 50 mM ammonium bicarbonate was added to the gel cubes, and proteins were in gel digested overnight. The resulting peptides were extracted from the gel pieces by sonication in 2% formic acid (FA) in 60% ACN. The extracts were then collected, and this step was repeated three more times. A final gel dehydration step (i.e., sonication with 100% ACN) was used to minimize peptide loss. Peptide digests corresponding to the same band were combined and concentrated via speed-vac.

Concentrated in-gel digests were reconstituted in 0.1% FA and 5% ACN and injected onto a reverse phase column (C18, Michrom Magic—C18AQ-5μ 200 Å 0.1×150 mm) using a Tempo MDLC system (AB Sciex, Foster City, Calif.) coupled to a quadrupole-time of flight MS/MS Q-Star Elite mass spectrometer (AB Sciex). Peptides were loaded onto the column using 98% solvent A (5% ACN, 0.1% FA in water) and 2% solvent B (95% ACN, 0.1% FA in water) for 30 min and separated by a 130 min linear gradient of increasing solvent B by 0.37%/min to a final concentration of 50%. MS and MS/MS peptide spectra were acquired using information dependent acquisition (IDA). A mass range of 350-1600 Da was monitored in TOF MS scan. The three most abundant precursor ions from TOF MS scans with an intensity >20 counts per second were submitted for MS/MS analyses. Former target ions were excluded from MS/MS submission for 15 s. MS data were acquired using Analyst QS (AB Sciex), and tandem mass spectra were extracted by mascot.dll and analyzed using Mascot (Matrix Science, London, UK; Mascot Server version 2.4.1). Mascot was set up to search three in house databases: 1: contaminants 20120713 (247 sequences; 128,130 residues), 2: cRAP 20121128 (112 sequences; 37,418 residues), and 3: VEEV database (6 sequences; 1,980 residues). Common contaminants were included in the first two databases while the complete VEEV protease, thioredoxin, complete sequence of CFP-TRIM14-YFP, as well as its predicted N-terminal and C-terminal sequences as produced by VEEV. Assuming the digestion was semitryptic (at least one peptide terminal was R or K) and allowing for 3 miscleavages. Fragment ion mass tolerance was set to 0.20 Da and a parent ion tolerance to 0.20 Da. Deamidation of asparagine and glutamine, oxidation of methionine were set as variable modifications. After identification by Mascot, the spectra of resulting N-terminal and C-terminal peptides of TRIM14 products from VEEV proteolysis: HYWEVDVQEAGA (SEQ ID No: 7) and GWWVGAMVS (SEQ ID No: 8), respectively) were inspected manually in the raw acquired data, and the resulting singly charged fragments were manually annotated

Western Blotting.

Cells were lysed in RIPA buffer containing Halt Protease Inhibitor Cocktail at a 2× final concentration. Lysates were separated in a 10% NuPAGE Bis-Tris gel and electroblotted onto a nitrocellulose membrane using the iBlot system (Invitrogen). Following protein transfer, blots were blocked in 1×PBS containing 0.05% Tween-20 and 5% dry milk and incubated at 4° C. overnight. Protein-specific primary antibodies were diluted in blocking buffer and incubated at RT for 2 hrs. Following incubation, blots were washed 3 times with PBS containing 0.05% Tween-20 (PBST). After washing blots were incubated with corresponding secondary antibody at RT for 1 hr then washed 3 times with PBST. For protein detection, blots were treated with SuperSignal™ West Pico Chemiluminescent Substrate and imaged using BioRad imaging software. Trim14 protein was detected using a polyclonal anti-Trim14 Ab (1:500, HPA053217) followed by goat anti-rabbit Horseradish peroxidase (HRP, 1:500) secondary Ab. Actin protein was detected using anti-actin Ab (1:5000) followed by goat anti-mouse HRP (1:5000) secondary Ab. The VEEV nsP2 protein was detected using goat anti-VEEV nsP2 Ab (kind gift from AlphaVax, Research Triangle Park, N.C., 1:1000) followed by rabbit anti-goat HRP (1:5000) secondary Ab.

A549 cells (adenocarcinoma human alveolar basal epithelial cells) were used. Infected A549 cell lysates collected at 6 and 24 h post-infection (10 μg/lane) were separated in a 10% NuPAGE Bis-Tris gel and transferred onto a nitrocellulose membrane. Trim14-α, Trim14-α cleavage product (CP), and α-actin were detected by Western blot analysis using protein specific antibodies. Recombinant Human Trim 14 protein was used as control. The VEEV Trinidad, EEEV FL93-939, WEEV CBA87, and CHIKV AF15561 viruses were used.

To test the effects of a previously identified VEEV nsP2 cysteine protease inhibitor (42), CA074 methylester (CA074me), A549 cells were treated with CA074me and infected at a multiplicity of infection equal to 10 with VEEV or CHIKV. After incubation of virus with cells for 1 h, cell monolayers were washed twice with medium to remove residual virus. Complete medium containing CA074me (50, 100, 200 μM) was added, and the cells were incubated at 37° C., 5% CO₂. At 18-24 h post-infection, supernatants and cell lysates were collected for analysis by western blot.

The specificity of the polyclonal rabbit Sigma Prestige™ anti-TRIM14 antibody (HPA053217) has already been analyzed and is available online (38). The HPA053217 antibody had been raised using an N-terminal sequence is common to full-length TRIM14 and the α- and β-isoforms of TRIM14. The sequence precedes the ubiquitination site

Modeling of Substrate Binding Interaction.

The binding models of substrates including VEEV P12, P23, P34 and TRIM14 were predicted with an ensemble-docking protocol using the AutoDock program (89). Multiple conformations of the VEEV nsP2 structure (PDB 2HWK) and the CHIKV nsP2 (PDB 3TRK) were obtained from MD simulations and cluster analysis. The active site of the protein was defined by a grid of 70×70×70 points with a grid spacing of 0.375 Å centered at the catalytic residue Cys-477. The Lamarckian Genetic Algorithm (LGA) was applied with 50 runs, and the best pose with the most favorable binding free energy was selected. MD simulations were performed for the predicted substrate binding models using the AMBER 12 package and the ff99SB force field. The solvated systems were subjected to a thorough energy minimization prior to MD simulations. Periodic boundary conditions were applied to simulate a continuous system. The particle mesh Ewald (PME) method was employed to calculate the long-range electrostatic interactions. The simulated system was first subjected to a gradual temperature increase from 0 K to 300 K over 100 ps, and then equilibrated for 500 ps at 300 K, followed by production runs of 2-ns length in total. The binding free energies were calculated using the MM-PBSA method. Decomposition of the calculated binding free energies was performed using the same MM-PBSA module in AMBER 12 package.

Detection of Infection

The VEEV-specific cleavage of TRIM14 could be used as a diagnostic biomarker of VEEV infection. VEEV/EEEV/WEEV and CHIKV all have similar symptoms, and currently there are no known biomarkers for VEEV-infections.

For example, material (such as blood or tissue) from an individual could be assayed for the possible presence of a product of the VEEV-specific cleavage from TRIM14 in order to determine whether or not the patient might be infected with VEEV. Such an assay can be performed using any suitable technique, for example immunohistochemistry (IHC), enzyme linked-immunosorbent assay (ELISA), mass spectrometry, and/or flow cytometry.

At least eight other Group IV (+)ssRNA viral proteases have been shown to cleave components of the MAVS signalosome to antagonize IFN production, suggesting that the assimilation of these short cleavage site motif sequences to host protein sequences may represent an embedded mechanism of IFN antagonism. Thus, it is expected that the technique could be used to detect host-pathogen interactions during infection by other members of this viral family. For instance, the method was used to identify potential host protein targets that may beresponsible for microcephaly in Zika virus infections.

Such a technique could be incorporated into a diagnostic assay or predictive software program.

Proteome Editing

Also contemplated is a protein analog to CRISPR/Cas9 and RNAi/RISC. This system relies on the short stretches of homologous host-pathogen protein sequences (SSHHPS) and a protease (as opposed to a nuclease) that cleaves them.

The viral genome provides a delivery vehicle for the RNA encoding a wild type or mutated nsP2 protease directly into the cytoplasm (as opposed to endosomal vesicles). The catalytic nature of the protease may allow it to turnover many substrates within a cell. Replication of mutant or wild type viruses would offer a mechanism to transiently propagate the effects. This type of proteome editing method has not been exploited previously, and has the potential for therapeutic application.

In one embodiment, a host cell or organism expresses a recombinant viral nonstructural polyprotein that incorporates the homologous sequence acted upon by the VEEV nsP2 protease. Introduction of the virus to the cell or organism results in cleavage of the sequence in the polyprotein and host protein which can lead to loss of function of the protein that is cleaved.

In a further embodiment, the nsP2 protease is mutated to act upon an amino acid sequence of interest (different from the homologous host-pathogen protein sequence), so that the introduction of a virus carrying the mutated protease results in proteolysis of the desired target.

Advantages and New Features

Viral nsP proteases could be mutated or used as-is to recognize other host protein sequences to proteolytically shut-off cascades that lead to gene expression or to proteolyze a single protein. Embodiments can include introducing a wild type or modified protease into cells in vitro or in vivo (the cells including, for example, cell culture, tissue culture, and/or living animals optionally including humans) using techniques available in the art such as transfection, transgenics, infection with wild-type or genetically engineered virus, etc. Optionally, one or more genetically engineered or wild-type targets for the protease can be introduced as well. This strategy may be useful to kill tumor cells where oncogene expression has already taken place or for removing protein toxins. Other applications can include therapy to treat or prevent various disease, research into viral infection, and other situations where it can be desirable to cleave proteins within cells.

Alphaviruses can infect a variety of cell types and are pantropic. These viruses cause transient acute viral infections, and attenuated alphaviruses are currently in use for vaccination. The mutations that attenuate the TC-83 vaccine strain do not affect protease activity of the nsP2 cysteine protease. Some alphaviruses like VEEV are also able to cross the blood-brain barrier. The virion may serve as a useful delivery vehicle for RNA and for proteases to the brain.

### CONCLUDING REMARKS

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

TABLE 2
Group IV viral proteases that have been shown to cleave host-proteins
involved in the innate immune response. In bold are the proteins involved in IFN
production pathways. In red are the residues that are identical to a single viral
polyprotein cleavage site, in green are residues which are found at the same
position in other viral polyprotein cleavage sites. Other host protein targets may be
important for transcriptional and translational shut-off

| | | Viral | Cleavage Site | Host Protein | Cleavage site in |
|---|---|---|---|---|---|
| Virus | Family | protease | Motif | Substrate | Host protein |
| Poliovirus | Picomaviridae | 3Cpro | (QE)↓(LIGS) | RIG-1 | LKKFPQ↓GQKGKV¹ |
| | | | | TATA-binding Protein | QGLASPQ↓GAMTPG |
| | | | | TATA-binding Protein | AAAVQQ↓STSQQA |
| | | | | Poly(A)-binding protein | VHVQ↓GQ |
| | | | | (PABP) | |
| | | | | eIF5B | VMEQ↓G |
| | | 2Apro | | TATA-binding Protein | MMPY↓GTGLTP |
| Rhinovirus | | 3Cpro | (AV)XXQ↓G | NF-_{K}B | LLNQ↓GIP |
| type 1a | | | | eIF5B | VMEQ↓G |
| Echovirus | | | | NF-_{K}B | LLNQ↓GIP |
| type 1 | | | | | |
| Coxsackie | | | | NF-_{K}B | LLNQ↓GIP |
| B virus | | | | eIF5B | VMEQ↓G |
| Foot and | | 3Cpro | (QE)↓(LIGS) | NEMO | LALPSQ↓RRSPPE |
| Mouth | | | | eIF4A | TNVRAE↓VQKLQM |
| disease | | | | Histone H3 | PRKQL↓ATKAA |
| Virus | | leader | | eIF4G | SFANLG↓RTTLST |
| (FMDV) | | protease | | | |
| Hepatitis | | 3Cpro | (LVI)X(TSA)(QEX)↓ | NEMO | PVLKAQ↓ADIYK |
| A virus | | 3ABC | XXXX | MAVS | LASQ↓VDSP |
| | | 3CD | | TRIF | DWSQ↓GCSL |
| | | | | TRIF | IREQSQ↓HLDG |
| Dengue | Flaviviridae | ns2B/ns3 | QKKKQR↓SGVLWD | STING(MITA) | VRACLGCPLRR↓GALLLLSIY |
| Hepatitis | Flaviviridae | ns3/4A | C↓(SA) | MAVS | EREVPC↓HRPS |
| V Virus | | | | TRIF | PPPPPSSTPC↓SAHLTPSSLE |
| VEEV | Togaviridae | nsP2 | AG(ACR)↓(GAY) | TRIM14 | DCFATGRHYWEVDVQEAGA↓ |
| | | | | | GWWVGA |

¹Based upon biochemical observations (e.g. MW of cleavage products) and sequence similarity to the motif

### REFERENCES

1. Steele, K. E., Reed, D., Glass, P. J., Hart, M. K., Ludwig, G. V., Pratt, W D., Parker, M. D., and Smith, J. F. (2007) Alphavirus Encephalitides, in Medical Aspects of Biological Warfare (Dembek, Z. F., Ed.) pp 241-270, Office of the Surgeon General, Falls Church.
2. Schafer, A., Brooke, C. B., Whitmore, A. C., and Johnston, R. E. (2011) The role of the blood-brain barrier during Venezuelan equine encephalitis virus infection, J Virol. 85, 10682-10690.
3. Ryzhikov, A. B., Tkacheva, N. V., Sergeev, A. N., and Ryabchikova, E. I. (1991) Venezuelan equine encephalitis virus propagation in the olfactory tract of normal and immunized mice, Biomed. Sci. 2, 607-614.
4. Steele, K. E. and Twenhafel, N. A. (2010) REVIEW PAPER: pathology of animal models of alphavirus encephalitis, Vet. Pathol. 47, 790-805.
5. Steele, K. E., Reed, D., Glass, P. J., Hart, M. K., Ludwig, G. V., Pratt, W D., Parker, M. D., and Smith, J. F. (2007) Alphavirus Encephalitides, in Medical Aspects of Biological Warfare (Dembek, Z. F., Ed.) pp 241-270, Office of the Surgeon General, Falls Church.
6. Hanson, R. P., Sulkin, S. E., Beuscher, E. L., Hammon, W M., MCKINNEY, R. W, and Work, T. H. (1967) Arbovirus infections of laboratory workers. Extent of problem emphasizes the need for more effective measures to reduce hazards, Science 158, 1283-1286.
7. Ehrlich, R. and Miller, S. (1971) Effect of relative humidity and temperature on airborne Venezuelan equine encephalitis virus, Appl. Microbiol. 22, 194-199.
8. Zacks, M. A. and Paessler, S. (2010) Encephalitic alphaviruses, Vet. Microbiol. 140, 281-286.
9. Johnson, K. M. and Martin, D. H. (1974) Venezuelan equine encephalitis, Adv. Vet. Sci. Comp Med 18, 79-116.
10. Deresiewicz, R. L., Thaler, S. J., Hsu, L., and Zamani, A. A. (1997) Clinical and neuroradiographic manifestations of eastern equine encephalitis, N Engl J Med 336, 1867-1874.
11. (2009) Encyclopedia of microbiology Elsevier, New York.
12. Fields, B. N., Knipe, D. M., and Howley, P. M. (2007) Fields' virology Wolters Kluwer Health/Lippincott Williams & Wilkins, Philadelphia.
13. Yin, J., Gardner, C. L., Burke, C. W, Ryman, K. D., and Klimstra, W. B. (2009) Similarities and differences in antagonism of neuron alpha/beta interferon responses by Venezuelan equine encephalitis and Sindbis alphaviruses, J Virol. 83, 10036-10047.
14. Garmashova, N., Gorchakov, R., Volkova, E., Paessler, S., Frolova, E., and Frolov, I. (2007) The Old World and New World alphaviruses use different virus-specific proteins for induction of transcriptional shutoff, J Virol. 81, 2472-2484.
15. Hahn, C. S., Lustig, S., Strauss, E. G., and Strauss, J. H. (1988) Western equine encephalitis virus is a recombinant virus, Proc Natl Acad Sci USA 85, 5997-6001.
16. Furr, M. and Reed, S. (2015) Equine Neurology Wiley, Somerset.
17. Akhrymuk, I., Frolov, I., and Frolova, E. I. (2016) Both RIG-I and MDAS detect alphavirus replication in concentration-dependent mode, Virology 487, 230-241.
18. Nikonov, A., Molder, T., Sikut, R., Kiiver, K., Mannik, A., Toots, U., Lulla, A., Lulla, V., Utt, A., Merits, A., and Ustav, M. (2013) RIG-I and MDA-5 detection of viral RNA-dependent RNA polymerase activity restricts positive-strand RNA virus replication, PLoS Pathog. 9, e1003610.
19. Kell, A. M. and Gale, M., Jr. (2015) RIG-I in RNA virus recognition, Virology 479-480, 110-121.
20. Frolova, E. I., Fayzulin, R. Z., Cook, S. H., Griffin, D. E., Rice, C. M., and Frolov, I. (2002) Roles of nonstructural protein nsP2 and Alpha/Beta interferons in determining the outcome of Sindbis virus infection, J Virol. 76, 11254-11264.
21. Zhang, Y, Burke, C. W, Ryman, K. D., and Klimstra, W B. (2007) Identification and characterization of interferon-induced proteins that inhibit alphavirus replication, J Virol. 81, 11246-11255.
22. Schoggins, J. W. and Rice, C. M. (2011) Interferon-stimulated genes and their antiviral effector functions, Curt Opin. Virol. 1, 519-525.
23. Simmons, J. D., White L J FAU—Morrison, T., Morrison T E FAU Montgomery, S., Montgomery S A FAU—Whitmore, A., Whitmore A C FAU—Johnston, R., Johnston R E FAU—Heise, M., and Heise, M. T. Venezuelan equine encephalitis virus disrupts STAT1 signaling by distinct mechanisms independent of host shutoff.
24. Schoggins, J. W. (2014) Interferon-stimulated genes: roles in viral pathogenesis, Curr. Opin. Virol. 6, 40-46.
25. Hollidge, B. S., Weiss, S. R., and Soldan, S. S. (2011) The role of interferon antagonist, non-structural proteins in the pathogenesis and emergence of arboviruses, Viruses. 3, 629-658.
26. Akhrymuk, I., Kulemzin, S. V., and Frolova, E. I. (2012) Evasion of the innate immune response: the Old World alphavirus nsP2 protein induces rapid degradation of Rpb1, a catalytic subunit of RNA polymerase II, J Virol. 86, 7180-7191.
27. Garmashova, N., Gorchakov, R., Volkova, E., Paessler, S., Frolova, E., and Frolov, I. (2007) The Old World and New World alphaviruses use different virus-specific proteins for induction of transcriptional shutoff, J Virol. 81, 2472-2484.
28. Garmashova, N., Atasheva, S., Kang, W, Weaver, S. C., Frolova, E., and Frolov, I. (2007) Analysis of Venezuelan equine encephalitis virus capsid protein function in the inhibition of cellular transcription, J Virol. 81, 13552-13565.
29. Atasheva, S., Fish, A., Fornerod, M., and Frolova, E. I. (2010) Venezuelan equine Encephalitis virus capsid protein forms a tetrameric complex with CRM1 and importin alpha/beta that obstructs nuclear pore complex function, J Virol. 84, 4158-4171.
30. Hu, X., Compton, J. R., Leary, D. H., Olson, M. A., Lee, M. S., Cheung, J., Ye, W, Ferrer, M., Southall, N., Jadhav, A., Morazzani, E. M., Glass, P. J., Marugan, J., and Legler, P. M. (2016) Kinetic, Mutational, and Structural Studies of the Venezuelan Equine Encephalitis Virus Nonstructural Protein 2 Cysteine Protease, Biochemistry 55, 3007-3019.
31. Hirose, S., Nishizumi, H., and Sakano, H. (2003) Pub, a novel PU.1 binding protein, regulates the transcriptional activity of PU.1, Biochem Biophys Res. Commun. 311, 351-360.
32. Zhou, Z., Jia, X., Xue, Q., Dou, Z., Ma, Y, Zhao, Z., Jiang, Z., He, B., Jin, Q., and Wang, J. (2014) TRIM14 is a mitochondrial adaptor that facilitates retinoic acid-inducible gene-I-like receptor-mediated innate immune response, Proc. Natl. Acad. Sci. U. S. A 111, E245-E254.
33. Nenasheva, V. V., Kovaleva G V, F. A. U., Uryvaev L V, F. A. U., Ionova K S, F. A. U., dova A V, F. A. U., Vorkunova G K, F. A. U., Chernyshenko S V, F. A. U., Khaidarova N V, F. A. U., and Tarantul, V. Z. Enhanced expression of trim14 gene suppressed Sindbis virus reproduction and modulated the transcription of a large number of genes of innate immunity.
34. Balistreri, G., Caldentey, J., Kaariainen, L., and Ahola, T. (2007) Enzymatic defects of the nsP2 proteins of Semliki Forest virus temperature-sensitive mutants, J Virol. 81, 2849-2860.
35. Schechter, I. and Berger, A. (1967) On the size of the active site in proteases. I. Papain., Biochem. Biophys. Res. Commun. 27, 157-162.
36. Altschul, S. F., Gish, W, Miller, W, Myers, E. W, and Lipman, D. J. (1990) Basic local alignment search tool, J Mol. Biol. 215, 403410.
37. Strauss, E. G., De Groot, R. J., Levinson, R., and Strauss, J. H. (1992) Identification of the active site residues in the nsP2 proteinase of Sindbis virus, Virology 191, 932-940.
38. Uhlen, M., Fagerberg, L., Hallstrom, B. M., Lindskog, C., Oksvold, P., Mardinoglu, A., Sivertsson, A., Kampf, C., Sjostedt, E., Asplund, A., Olsson, I., Edlund, K., Lundberg, E., Navani, S., Szigyarto, C. A., Odeberg, J., Djureinovic, D., Takanen, J. O., Hober, S., Alm, T., Edqvist, P. H., Berling, H., Tegel, H., Mulder, J., Rockberg, J., Nilsson, P., Schwenk, J. M., Hamsten, M., von, F. K., Forsberg, M., Persson, L., Johansson, F., Zwahlen, M., von, H. G., Nielsen, J., and Ponten, F. (2015) Proteomics. Tissue-based map of the human proteome, Science 347, 1260419.
39. Jia, X., Zhou, H., Wu, C., Wu, Q., Ma, S., Wei, C., Cao, Y, Song, J., Zhong, H., Zhou, Z., and Wang, J. (2017) The Ubiquitin Ligase RNF125 Targets Innate Immune Adaptor Protein TRIM14 for Ubiquitination and Degradation, J Immunol 198, 4652-4658.
40. Wang, S., Chen, Y, Li, C., Wu, Y, Guo, L., Peng, C., Huang, Y., Cheng, G., and Qin, F. X. TRIM14 inhibits hepatitis C virus infection by SPRY domain-dependent targeted degradation of the viral NSSA protein.
41. Carthagena, L., Bergamaschi, A., Luna, J. M., David, A., Uchil, P. D., Margottin-Goguet, F., Mothes, W, Hazan, U., Transy, C., Pancino, G., and Nisole, S. A. (2009) Human TRIM Gene Expression in Response to Interferons, PLoS One 4, e4894.
42. Campos-Gomez, J., Ahmad, F., Rodriguez, E., and Saeed, M. F. (2016) A novel cell-based assay to measure activity of Venezuelan equine encephalitis virus nsP2 protease, Virology 496, 77-89.
43. D'Cruz, A. A., Babon, J. J., Norton, R. S., Nicola, N. A., and Nicholson, S. E. (2013) Structure and function of the SPRY/B30.2 domain proteins involved in innate immunity, Protein Sci 22, 1-10.
44. James, L. C., Keeble, A. H., Khan, Z., Rhodes, D. A., and Trowsdale, J. (2007) Structural basis for PRYSPRY-mediated tripartite motif (TRIM) protein function, Proc Natl Acad Sci USA 104, 6200-6205.
45. Ozato, K., Shin, D. M., Chang, T. H., and Morse, H. C., III (2008) TRIM family proteins and their emerging roles in innate immunity, Nat Rev Immunol 8, 849-860.
46. Vaysburd, M., Watkinson, R. E., Cooper, H., Reed, M., O'Connell, K., Smith, J., Cruickshanks, J., and James, L. C. (2013) Intracellular antibody receptor TRIM21 prevents fatal viral infection, Proc Natl Acad Sci USA 110, 12397-12401.
47. Di, P. A., Kajaste-Rudnitski, A., Oteiza, A., Nicora, L., Towers, G. J., Mechti, N., and Vicenzi, E. (2013) TRIM22 inhibits influenza A virus infection by targeting the viral nucleoprotein for degradation, J Virol. 87, 4523-4533.
48. Everett, R. D. and Chelbi-Alix, M. K. (2007) PML and PML nuclear bodies: implications in antiviral defence, Biochimie 89, 819-830.
49. Ozato, K., Shin, D. M., Chang, T. H., and Morse, H. C., III (2008) TRIM family proteins and their emerging roles in innate immunity, Nat Rev Immunol 8, 849-860.
50. James, L. C., Keeble, A. H., Khan, Z., Rhodes, D. A., and Trowsdale, J. (2007) Structural basis for PRYSPRY-mediated tripartite motif (TRIM) protein function, Proceedings of the National Academy of Sciences 104, 6200-6205.
51. Malim, M. H. and Bieniasz, P. D. (2012) HIV Restriction Factors and Mechanisms of Evasion, Cold Spring Harb. Perspect Med 2, a006940.
52. Nisole, S., Stoye, J. P., and Saib, A. (2005) TRIM family proteins: retroviral restriction and antiviral defence, Nat Rev Microbiol. 3, 799-808.
53. Carthagena, L., Bergamaschi, A., Luna, J. M., David, A., Uchil, P. D., Margottin-Goguet, F., Mothes, W, Hazan, U., Transy, C., Pancino, G., and Nisole, S. (2009) Human TRIM gene expression in response to interferons, PLoS One 4, e4894.
54. Bhoj, V. G., Sun, Q., Bhoj, E. J., Somers, C., Chen, X., Tones, J. P., Mejias, A., Gomez, A. M., Jafri, H., Ramilo, O., and Chen, Z. J. (2008) MAVS and MyD88 are essential for innate immunity but not cytotoxic T lymphocyte response against respiratory syncytial virus, Proc Natl Acad Sci USA 105, 14046-14051.
55. Mibayashi, M., Martinez-Sobrido, L. F., Loo Y M FAU—Cardenas, W, Cardenas W B FAU—Gale, M. J., Gale, M., Jr., and Garcia-Sastre, A. Inhibition of retinoic acid-inducible gene I-mediated induction of beta interferon by the NS1 protein of influenza A virus.
56. Kundu, P., Raychaudhuri, S., Tsai, W, and Dasgupta, A. (2005) Shutoff of RNA polymerase II transcription by poliovirus involves 3C protease-mediated cleavage of the TATA-binding protein at an alternative site: incomplete shutoff of transcription interferes with efficient viral replication, J Virol. 79, 9702-9713.
57. Das, S. and Dasgupta, A. (1993) Identification of the cleavage site and determinants required for poliovirus 3CPro-catalyzed cleavage of human TATA-binding transcription factor TBP, J Virol. 67, 3326-3331.
58. Weidman, M. K., Sharma, R., Raychaudhuri, S., Kundu, P., Tsai, W, and Dasgupta, A. (2003) The interaction of cytoplasmic RNA viruses with the nucleus, Virus Res. 95, 75-85.
59. Kuyumcu-Martinez, N. M., Van Eden, M. E., Younan, P., and Lloyd, R. E. (2004) Cleavage of poly(A)-binding protein by poliovirus 3C protease inhibits host cell translation: a novel mechanism for host translation shutoff, Mol Cell Biol 24, 1779-1790.
60. Kuyumcu-Martinez, N. M., Joachims, M., and Lloyd, R. E. (2002) Efficient cleavage of ribosome-associated poly(A)-binding protein by enterovirus 3C protease, J Virol. 76, 2062-2074.
61. de, B. S., Bonderoff, J. M., Chumakov, K. M., Lloyd, R. E., and Hellen, C. U. (2008) Cleavage of eukaryotic initiation factor eIF5B by enterovirus 3C proteases, Virology 378, 118-122.
62. Li, W, Ross-Smith, N., Proud, C. G., and Belsham, G. J. (2001) Cleavage of translation initiation factor 4AI (eIF4AI) but not eIF4AII by foot-and-mouth disease virus 3C protease: identification of the eIF4AI cleavage site, FEBS Lett 507, 1-5.
63. Foeger, N., Glaser, W, and Skern, T. (2002) Recognition of eukaryotic initiation factor 4G isoforms by picornaviral proteinases, J Biol Chem 277, 44300-44309.
64. Qu, L., Feng, Z., Yamane, D., Liang, Y., Lanford, R. E., Li, K., and Lemon, S. M. (2011) Disruption of TLR3 signaling due to cleavage of TRIF by the hepatitis A virus protease-polymerase processing intermediate, 3CD, PLoS Pathog. 7, e1002169.
65. Banal, P. M., Sarkar, D., Fisher, P. B., and Racaniello, V. R. (2009) RIG-I is cleaved during picornavirus infection, Virology 391, 171-176.
66. Banal, P. M., Morrison, J. M., Drahos, J., Gupta, P., Sarkar, D., Fisher, P. B., and Racaniello, V. R. (2007) MDA-5 is cleaved in poliovirus-infected cells, J Virol. 81, 3677-3684.
67. Yang, Y., Liang, Y., Qu, L., Chen, Z., Yi, M., Li, K., and Lemon, S. M. (2007) Disruption of innate immunity due to mitochondrial targeting of a picornaviral protease precursor, Proc Natl Acad Sci USA 104, 7253-7258.
68. Neznanov, N., Chumakov, K. M., Neznanova, L., Almasan, A., Banerjee, A. K., and Gudkov, A. V. (2005) Proteolytic cleavage of the p65-RelA subunit of NF-kappaB during poliovirus infection, J Biol Chem 280, 24153-24158.
69. Wang, D., Fang, L., Li, K., Zhong, H., Fan, J., Ouyang, C., Zhang, H., Duan, E., Luo, R., Zhang, Z., Liu, X., Chen, H., and Xiao, S. (2012) Foot-and-mouth disease virus 3C protease cleaves NEMO to impair innate immune signaling, J Virol. 86, 9311-9322.
70. Wang, D., Fang, L., Wei, D., Zhang, H., Luo, R., Chen, H., Li, K., and Xiao, S. (2014) Hepatitis A virus 3C protease cleaves NEMO to impair induction of beta interferon, J Virol. 88, 10252-10258.
71. Lin, R., Lacoste, J., Nakhaei, P., Sun, Q., Yang, L., Paz, S., Wilkinson, P., Julkunen, I., Vitour, D., Meurs, E., and Hiscott, J. (2006) Dissociation of a MAVS/IPS-1/VISA/Cardif-IKKepsilon molecular complex from the mitochondrial outer membrane by hepatitis C virus NS3-4A proteolytic cleavage, J Virol. 80, 6072-6083.
72. Hiscott, J., Lacoste, J., and Lin, R. (2006) Recruitment of an interferon molecular signaling complex to the mitochondrial membrane: disruption by hepatitis C virus NS3-4A protease, Biochem. Pharmacol. 72, 1477-1484.
73. Bellecave, P., Sarasin-Filipowicz, M., Donze, O., Kennel, A., Gouttenoire, J., Meylan, E., Terracciano, L., Tschopp, J., Sarrazin, C., Berg, T., Moradpour, D., and Heim, M. H. (2010) Cleavage of mitochondrial antiviral signaling protein in the liver of patients with chronic hepatitis C correlates with a reduced activation of the endogenous interferon system, Hepatology 51, 1127-1136.
74. Meylan, E., Curran, J., Hofmann, K., Moradpour, D., Binder, M., Bartenschlager, R., and Tschopp, J. (2005) Cardif is an adaptor protein in the RIG-I antiviral pathway and is targeted by hepatitis C virus, Nature 437, 1167-1172.
75. Yu, C. Y, Chang, T. H., Liang, J. J., Chiang, R. L., Lee, Y L., Liao, C. L., and Lin, Y. L. (2012) Dengue Virus Targets the Adaptor Protein MITA to Subvert Host Innate Immunity, PLoS Pathog 8, e1002780.
76. Aguirre, S., Maestre, A. M., Pagni, S., Patel, J. R., Savage, T., Gutman, D., Maringer, K., Bernal-Rubio, D., Shabman, R. S., Simon, V., Rodriguez-Madoz, J. R., Mulder, L. C. F., Barber, G. N., and Fernandez-Sesma, A. (2012) DENY Inhibits Type I IFN Production in Infected Cells by Cleaving Human STING, PLoS Pathog 8, e1002934.
77. Li, J., Lim S P FAU—Beer, D., Beer, D. F., Patel, V. F., Wen, D. F., Tumanut, C. F., Tully D C FAU—Williams, J., Williams J A FAU—Jiricek, J., Jiricek, J. F., Priestle J P FAU—Harris, J., Harris J L FAU—Vasudevan, S., and Vasudevan, S. G. Functional profiling of recombinant NS3 proteases from all four serotypes of dengue virus using tetrapeptide and octapeptide substrate libraries.
78. Blom, N., Hansen, J., Blaas, D., and Brunak, S. (1996) Cleavage site analysis in picornaviral polyproteins: discovering cellular targets by neural networks, Protein Sci 5, 2203-2216.
79. Mukherjee, A., Morosky, S. A., orme-Axford, E., Dybdahl-Sissoko, N., Oberste, M. S., Wang, T., and Coyne, C. B. (2011) The coxsackievirus B 3C protease cleaves MAVS and TRIF to attenuate host type I interferon and apoptotic signaling, PLoS Pathog. 7, e1001311.
80. Badorff, C., Berkely, N., Mehrotra, S., Talhouk, J. W, Rhoads, R. E., and Knowlton, K. U. (2000) Enteroviral protease 2A directly cleaves dystrophin and is inhibited by a dystrophin-based substrate analogue, J Biol Chem 275, 11191-11197.
81. Weidman, M. K., Sharma, R., Raychaudhuri, S., Kundu, P., Tsai, W, and Dasgupta, A. (2003) The interaction of cytoplasmic RNA viruses with the nucleus, Virus Res. 95, 75-85.
82. Falk, M. M., Grigera, P. R., Bergmann, I. E., Zibert, A., Multhaup, G., and Beck, E. (1990) Foot-and-mouth disease virus protease 3C induces specific proteolytic cleavage of host cell histone H3, J Virol 64, 748-756.
83. Yalamanchili, P., Banerjee, R., and Dasgupta, A. (1997) Poliovirus-encoded protease 2APro cleaves the TATA-binding protein but does not inhibit host cell RNA polymerase II transcription in vitro, J Virol 71, 6881-6886.
84. Donnelly, M. I., Zhou, M., Millard, C. S., Clancy, S., Stols, L., Eschenfeldt, W. H., Collart, F. R., and Joachimiak, A. (2006) An expression vector tailored for large-scale, high-throughput purification of recombinant proteins, Protein Expr. Purif. 47, 446-454.
85. Legler, P. M., Cai, M., Peterkofsky, A., and Clore, G. M. (2004) Three-dimensional solution structure of the cytoplasmic B domain of the mannitol transporter Ilmannitol of the Escherichia coli phosphotransferase system, J. Biol. Chem. 279, 39115-39121.
86. Cheung, J., Franklin, M., Mancia, F., Rudolph, M., Cassidy, M., Gary, E., Burshteyn, F., and Love, J. (2011) Structure of the Chikungunya virus nsP2 protease.
87. Dong, M., Tepp, W. H., Johnson, E. A., and Chapman, E. R. (2004) Using fluorescent sensors to detect botulinum neurotoxin activity in vitro and in living cells, Proc. Natl. Acad. Sci. U. S. A 101, 14701-14706.
88. Ruge, D. R., Dunning, F. M., Piazza, T. M., Molles, B. E., Adler, M., Zeytin, F. N., and Tucker, W. C. (2011) Detection of six serotypes of botulinum neurotoxin using fluorogenic reporters, Anal. Biochem. 411, 200-209.
89. Morris, G. M., Huey, R., Lindstrom, W, Sanner, M. F., Belew, R. K., Goodsell, D. S., and Olson, A. J. (2009) AutoDock4 and AutoDockTools4: Automated docking with selective receptor flexibility, J Comput. Chem. 30, 2785-2791.
90. Shin, G., Yost, S. A., Miller, M. T., Elrod, E. J., Grakoui, A., and Marcotrigiano, J. (2012) Structural and functional insights into alphavirus polyprotein processing and pathogenesis, Proc. Natl. Acad. Sci. U. S. A 109, 16534-16539.
91. James, L. C., Keeble, A. H., Khan, Z., Rhodes, D. A., and Trowsdale, J. (2007) Structural basis for PRYSPRY-mediated tripartite motif (TRIM) protein function, Proceedings of the National Academy of Sciences 104, 6200-6205.
92. (2017) Introduction to CRISPR and Cas9.
93. Hartig, J. S. (2017) Mechanism of siRNA silencing.

What is claimed is:

1. A method of cleaving a protein, comprising:
causing a cell to express a recombinant viral polyprotein of a Group IV virus that incorporates a cleavage site recognized by a protease; and
infecting the cell with the Group IV virus, thereby causing the viral protease to cleave the recombinant protein at the cleavage site.

2. The method of claim 1, wherein a plurality of cells in a living organism express the recombinant protein and wherein the living organism is infected with the virus.

3. The method of claim 1, wherein the Group IV virus is selected from the group consisting of poliovirus, rhinovirus type 1a, echovirus type 1, Coxsackie B virus, foot and mouth disease virus, hepatitis A virus, hepatitis C virus, dengue, Zika virus, and Venezuelan equine encephalitis virus.

4. The method of claim 1, wherein the Group IV virus is Venezuelan equine encephalitis virus.

5. The method of claim 1, wherein the recombinant protein is endogenous to the cell.

6. The method of claim 1, wherein the recombinant protein is exogenous to the cell.


SEQUENCE LISTING (8 sequences)

SEQ ID NO: 1 - protein, 6 aa, Homo sapiens
QEAGAG

SEQ ID NO: 2 - protein, 25 aa, Artificial Sequence
  synthetic construct
VEEPTLEADVDLMLQEAGAGSVETP

SEQ ID NO: 3 - protein, 25 aa, Artificial Sequence
  synthetic construct
TREEFEAFVAQQQRFDAGAYIFSSD

SEQ ID NO: 4 - protein, 25 aa, Artificial Sequence
  synthetic construct
DCFATGRHYWEVDVQEAGAGWWVGA

SEQ ID NO: 5 - protein, 22 aa, Artificial Sequence
  synthetic construct
ATGRHYWEVDVQEAGAGWWVGA

SEQ ID NO: 6 - protein, 19 aa, Artificial Sequence
  synthetic construct
RHYWEVDVQEAGAGWWVGA

SEQ ID NO: 7 - protein, 12 aa, Homo sapiens
HYWEVDVQEAGA

SEQ ID NO: 8 - protein, 9 aa, Homo sapiens
GWWVGAMVS